(19) United States
(12) Patent Application Publication
(10) Pub. No.: US 2010/0094076 A1
(43) Pub. Date: Apr. 15, 2010

(54) Connector for a cooling system for a magnetic stimulator

(75) Inventor: Mark Phillips, Pembrokeshire (GB)

Correspondence Address: NIXON & VANDERHYE, PC, 901 NORTH GLEBE ROAD, 11TH FLOOR, ARLINGTON, VA, 22203, US

(73) Assignee: THE MAGSTIM COMPANY LIMITED, Carmarthenshire (GB)

(21) Appl. No.: 12/461,344

(22) Filed: Aug. 7, 2009

(30) Foreign Application Priority Data
Aug. 7, 2008 (GB) 0814463.6

(51) Int. Cl. A61N 2/00; F28D 15/00

(52) U.S. Cl. 600/13; 165/104.33

(57) ABSTRACT

A magnetic neuro-muscular stimulator system, a stimulator coil of elongate conductor; a coolant system configured to cool the stimulated coil including a coolant jacket about at least a portion of the conductor; a connector for connecting the coolant system to the elongate conductor, the connector having a first port communicating with a first portion of the coolant system; a second port communicating with a second portion of the coolant system, the first and second ports being in fluid communication; and the connector further including a through bore such that a portion of the elongate conductor extends from externally of the connector through the bore and through the first port, wherein the elongate conductor is in sealed contact with a portion of the bore such that the coolant is prevented from flowing through the bore.

The present invention relates to magnetic stimulators, particularly for the magnetic stimulation of neuro muscular tissue. In particular, this invention relates to a component of a magnetic stimulator for magnetic stimulation of neuro muscular tissue.

Known magnetic stimulators generally comprise a charging circuit for a discharge capacitor, a discharge control such as a controlled rectifier for allowing the discharge of the capacitor through a stimulating coil, and other circuit elements for limiting the effect of undesirable electrical transients. A cooling system is also provided which drives coolant into proximity of the coil. For example, a pump may pump coolant into a jacket in which the coil is disposed. The coolant therefore flows about the coil and subsequently passes to a heat exchanger.

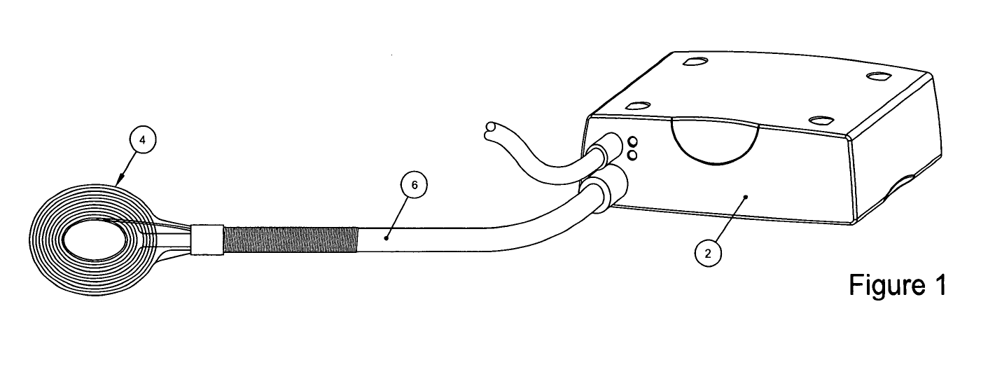

An example of a known magnetic stimulation device is schematically represented in FIG. 1 which shows a heat exchanger connection box 2 linked to coil 4 via conduit 6. Coolant having been cooled by the heat exchanger within box 2 is then pumped through a conduit around the coil and subsequently returned to the heat exchanger within the heat exchanger connection box 2.

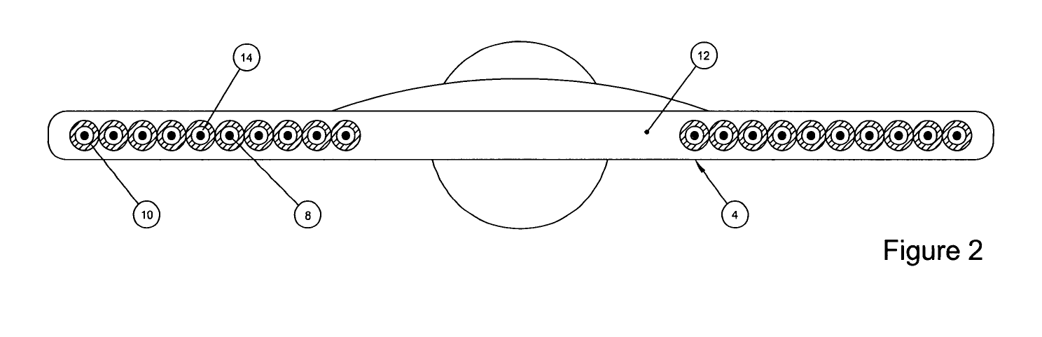

FIG. 2 is a schematic example of a cross section of the coiled conductor 4 showing the conductor 8 and surrounding jacket 10 encapsulated in a flexible sheath 12. The coolant can flow along the space 14 defined between the insulated conductor 8 and the surrounding jacket 10.

A problem with the arrangement as described is that a power supply is needed which charges a reserve capacitor which then discharges at appropriate intervals through the conductor. The conductor therefore has to enter into and exit the cooling circuit to pass to and from the cooling circuit to the power supply in order that the conductor can be cooled.

According to the present invention, there is a magnetic neuro-muscular stimulation system comprising:
a stimulator coil of elongate conductor;
a coolant system configured to cool the stimulated coil including a coolant jacket about at least a portion of the conductor;
a connector for connecting the coolant system to the elongate conductor, the connector having a first port communicating with a first portion of the coolant system; a second port communicating with a second portion of the coolant system, the first and second ports being in fluid communication; and the connector further including a through bore such that a portion of the elongate conductor extends from externally of the connector through the bore and through the first port, wherein the elongate conductor is in sealed contact with a portion of the bore such that the coolant is prevented from flowing through the bore.

The elongate conductor beneficially comprises a wire.

The elongate conductor is preferably sealed in the bore. The system preferably comprises a sealing arrangement for sealing the conductor in the bore of the connector.

The first and/or second port is beneficially defined by a nozzle, the nozzle arranged to connect to the first and/or second portion of the coolant jacket. Such a nozzle is therefore configured to locate within a conduit or tube of the coolant system and provide a seal there between. The nozzle is beneficially generally circular and has a diameter greater than the internal diameter of the first and/or second portion of the coolant jacket. The first and/or second portion of the coolant system preferably comprises a flexible elongate hollow member.

The nozzle beneficially has a portion arranged to engage with the first and/or second portion of the coolant system, the engaging portion having one or more protrusions thereon arranged to prevent accidental release of the first and/or second portion of the coolant system from the nozzle. Such protrusions provide a friction fit between the nozzle and coolant system.

The first portion and the second portion preferably each comprise a nozzle, the respective nozzles arranged to extend perpendicular to one another.

The nozzle is preferably releasably connected to the body of the connector.

The sealing arrangement beneficially comprises an elastically deformable material arranged to define at least a portion of the bore

The sealing arrangement may further comprise a cap arranged to be moveable against the elastically deformable material, to cause deformation of the elastically deformable material thereby reducing the cross sectional area of the bore

The sealing arrangement may further comprise a shoulder arranged to seat the elastically deformable material.

The cap is preferably arranged to be moveable relative to the shoulder thereby causing compression of the elastic material against the shoulder

The present invention will now be described by way of example only with reference to the accompanying drawings in which:

FIGS. 1 and 2 represent a prior art magnetic stimulator system.

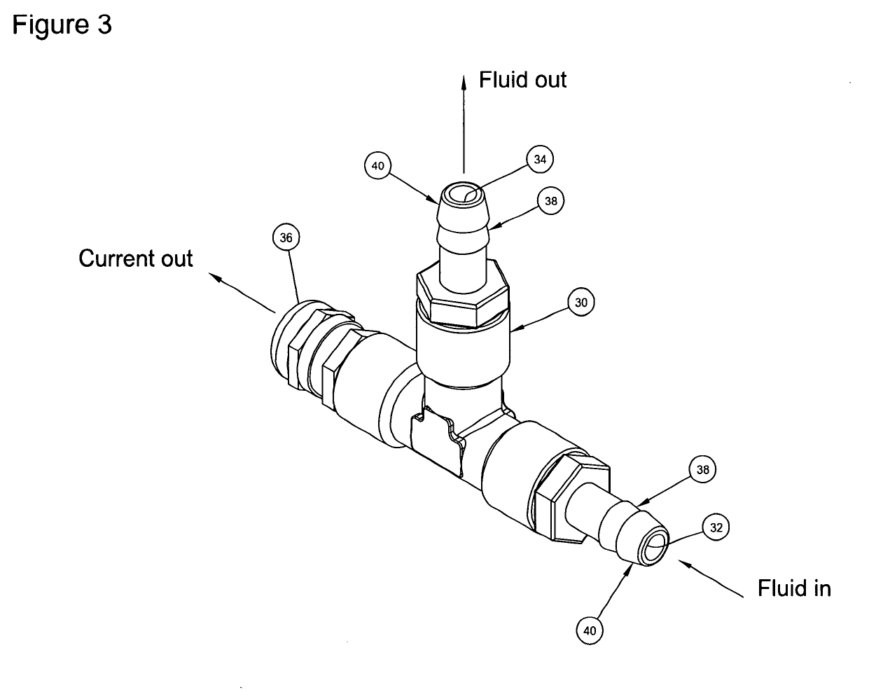

FIG. 3 is a schematic perspective view of a connector for a magnetic stimulator system.

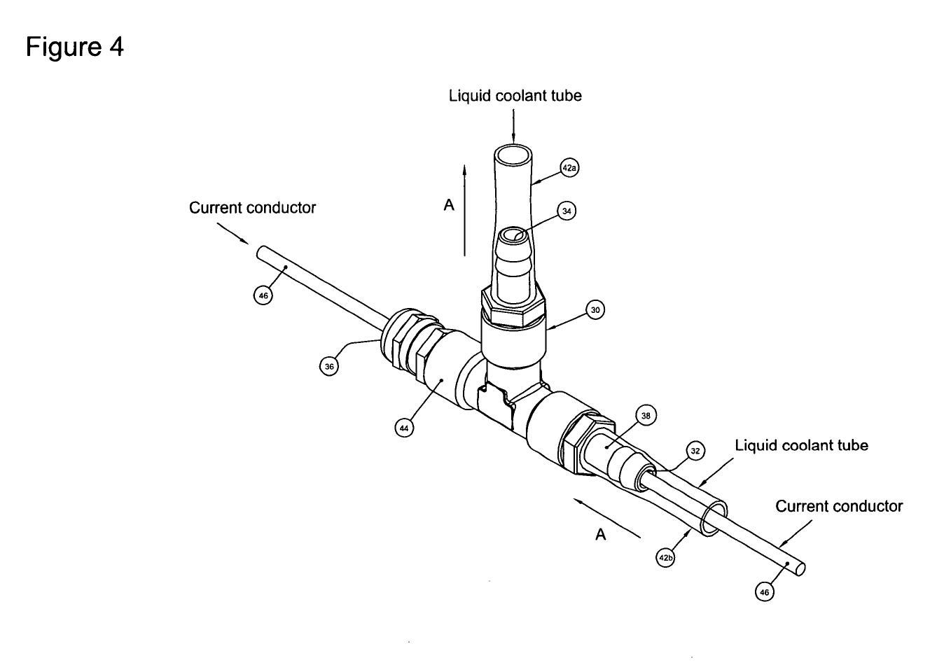

FIG. 4 is a schematic perspective view of a connector for a magnetic stimulator system in use showing a portion of the conductor wire and a portion of the coolant system.

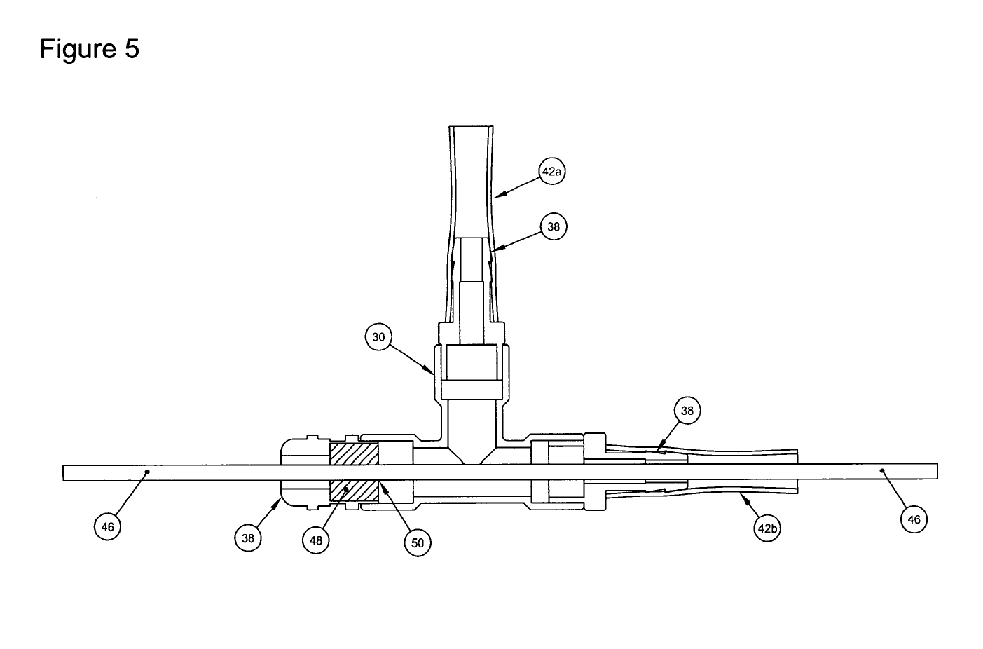

FIG. 5 is a schematic cross sectional view of the sealing arrangement arranged to prevent release of coolant from the passageway through which the conductor wire exits the connector.

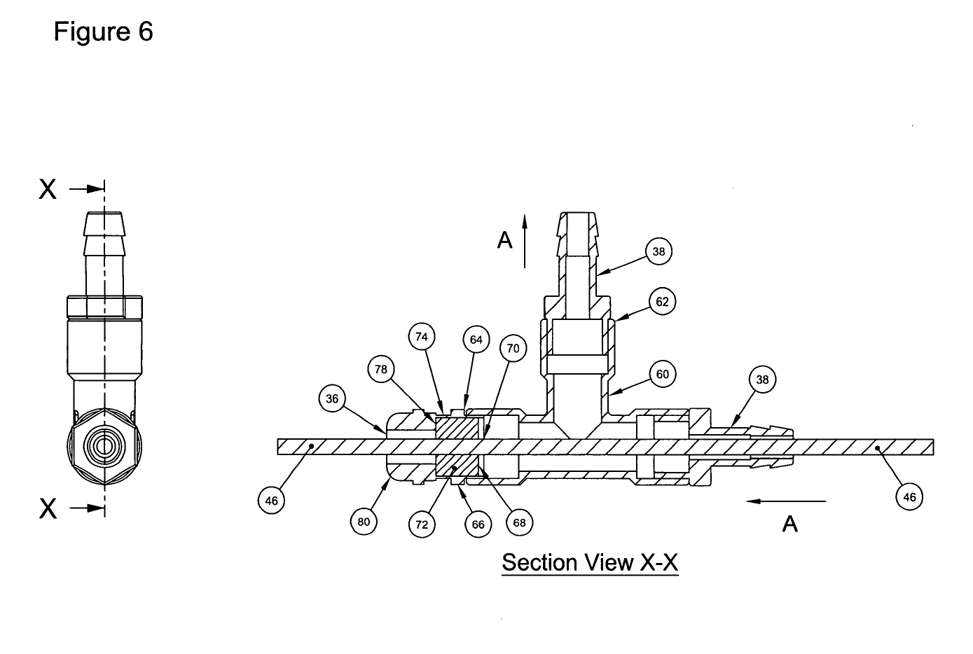

FIG. 6 is a schematic end and cross sectional view of a connector according to an exemplary embodiment of the present invention.

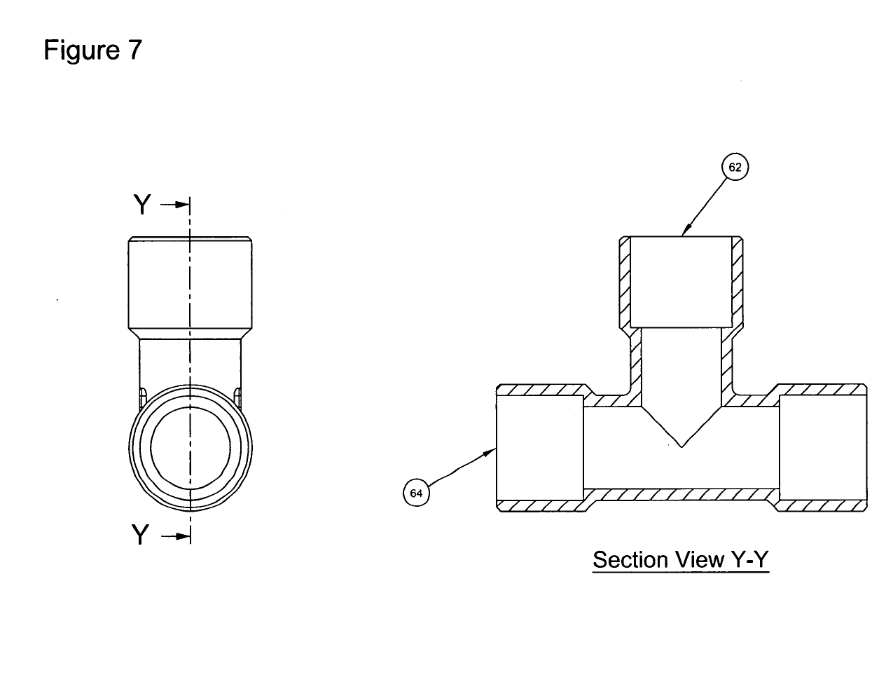

FIG. 7 is a schematic end and cross sectional view of a “T” piece of the connector according to an exemplary embodiment of the present invention.

Referring to FIG. 3, there is a connector 30 having a first port 32 and a second port 34 through which coolant flows. A third port 36 is provided which extends via a bore (not shown) into the connector, the channel extending through the connector 30 to the first port 32. First and second ports 32, 34 are defined by a nozzle arrangement 38 which seats within a coolant tube of the coolant system. A friction fit is provided between the nozzle 38 and the coolant tube. The friction fit is achieved as the coolant tube is flexible and the nozzle causes expansion of the coolant tube about the nozzle providing the friction fit and also providing a seal. Protrusions 40 are provided which act as a barb to reduce the chance of accidental release of the coolant tube from the nozzle. It will be appreciated that alternative connections between the connector and the coolant system may be provided, however, such a nozzle type arrangement is relatively simple to assemble and provides a reliable seal.

Referring to FIG. 4, the coolant tubes 42 (a) and (b) are shown connected to the nozzles 38 and show that the ends of the tubes communicating with the nozzles are elastically deformed about the nozzles to provide a seal there between. The conductor wire 46 passes through port 32, through the channel in the connector 30 and exits from the third port 36. A sealing arrangement (not shown) is beneficially provided within connector portion 44 which is shown in detail in FIG. 5. It will therefore be appreciated that the conductor wire 46 may pass through the connector 30 but coolant is subsequently prevented from passing through the third port 36, as indicated by arrows A. Coolant may therefore flow through coolant tube 42b, through the connector 30 and out through coolant tube 42a. The outer diameter of the conductor wire 46 is less than the diameter of the port 32 such that coolant can pass from the coolant tube 42b through nozzle 38 and out through port 34.

Referring to FIG. 5, a cross section of the connector portion is represented in particular showing the sealing arrangement 48. The sealing arrangement is arranged to prevent coolant from exiting the channel 50 and as indicated in FIG. 5 can be seen to locate about the conductor wire 46. It is envisaged that the conductor wire 46 can extend through the channel generally through the central portion of the channel 50, or at or adjacent an extremity of the channel 50. Beneficially, however, the conductor wire has an approximate diameter of about 2.5-3 mm, and the channel 50 therefore has a diameter greater than the diameter of the conductor wire such that assembly can be easily achieved by passing the conductor wire through the channel 50. The sealing arrangement 48 may be a bung made of an elastomeric material having an aperture therethrough for receipt of the conductor wire 46. The bung may be deformed into the connector 30 to form a fluid type seal to prevent coolant flow through port 38.

It will be appreciated that a number of alternative arrangements for sealing the port 38 about the wire 46 may be provided.

Referring to FIG. 6 there is a preferred embodiment for the connector according to an exemplary embodiment of the present. Components common to other figures have been indicated with common numbering. The connector 30 is generally composed of a component forming a “T” section 60 as shown in more detail in FIG. 7. The “T” section 60 forms the major component of the connector 30 and nozzle arrangements 38 are arranged to releasably connect to the “T” section 60. The nozzle arrangements 38 are beneficially threaded and engaged with the corresponding thread portion on an internal face of the “T” portion 60 through aperture 62. Engagement between the thread of the nozzle arrangement 38 and the corresponding “T” section 60 ensures a liquid tight fit. Fluid flow therefore passes through the nozzle arrangements 38 and through the “T” section 60 and out through the second nozzle arrangement 38 as indicated by arrows denoted by reference “A”. It will be appreciated that the port 36 is provided to allow the wire 46 to extend therethrough but correspondingly prevent liquid escaping therefrom. An outlet 64 defined by the “T” section 60 is provided into which a threaded component 66 is releaseably engaged on the corresponding thread on the internal surface of the “T” section about the opening. The component 66 provides a seat 68 having an aperture 70 therethrough arranged to receive the wire 46. A bung 72 comprising an elastic deformable material, for example rubber, is also arranged to seat about the wire 46 such that the wire 46 passes therethrough. The bung 72 therefore seats within the threaded component 66. The opposing end 74 of the threaded component 66 also comprises a threaded portion which is arranged to engage with cap 76. Corresponding threads on the inner surface of the cap and the threads on the threaded components 66 matingly engage and a shoulder 78 is provided by the cap which is arranged to force against the bung 72 as the cap is threaded onto the thread of the threaded component 66. As the cap is tightened, the force on the bung 72 increases thus increasing the elastic deformation and accordingly the force on the conductor wire 46. Clearly, an increase in force improves the seal provided. The cap 80 also defines an aperture therethrough for receipt of the conductor wire 46.

Alternative coolant systems are envisaged, for example whereby the coil of conductor wire is immersed in coolant in a jacket which encapsulates a plurality of coils of the conductor wire. Alternatively, for example, the jacket may encapsulate the conductor wire which is subsequently wound into a coil. The coolant system may further comprise a pump, heat exchanger and reservoir thereby providing a coolant flow circuit.

The present invention has been described by way of example only and it will be appreciated by a person skilled in the art that modifications and variations may be made without departing from the scope of the appended claims.

1. A magnetic neuro-muscular stimulator system comprising
a stimulator coil of elongate conductor;
a coolant system configured to cool the stimulated coil including a coolant jacket about at least a portion of the conductor;
a connector for connecting the coolant system to the elongate conductor, the connector having a first port communicating with a first portion of the coolant system; a second port communicating with a second portion of the coolant system, the first and second ports being in fluid communication; and the connector further including a through bore such that a portion of the elongate conductor extends from externally of the connector through the bore and through the first port, wherein the elongate conductor is in sealed contact with a portion of the bore such that the coolant is prevented from flowing through the bore.

2. A system according to claim 1, wherein the elongate conductor is sealed in the bore.

3. A system according to claim 1, wherein the connector comprises a sealing arrangement arranged to prevent fluid flowing through the bore.

4. A system according to claim 1, wherein the first and/or second port is defined by a nozzle, the nozzle arranged to connect to the first and/or second portion of the coolant jacket.

5. A system according to claim 4, wherein the nozzle is generally circular and has a diameter greater than the internal diameter of the first and/or second portion of the coolant jacket.

6. A system according to claim 1, wherein the first and/or second portion of the coolant jacket comprises a flexible elongate hollow member.

7. A system according to claim 6, wherein the nozzle has a portion arranged to engage with the first and/or second portion of the cooling system, the engaging portion having one or more protrusions thereon arranged to prevent accidental release of the first and/or second portion of the cooling system from the nozzle.

8. A system according to claim 4, wherein the first portion and the second portion each comprises a nozzle, the respective nozzles arranged to extend generally perpendicular to one another.

9. A system according to claim 4 wherein the nozzle is releasably connected to the body of the connector.

10. A system according to claim 1, wherein the passage and the first port extend along substantially the same axis relative to each other.

11. A system according to claim 3 wherein the sealing arrangement comprises an elastically deformable material arranged to define at least a portion of the bore.

12. A system according claim 11 wherein the sealing arrangement further comprises a cap arranged to be moveable against the elastically deformable material, to cause deformation of the elastically deformable material thereby reducing the cross sectional area of the bore.

13. A system according to claim 11 wherein the sealing arrangement further comprises a shoulder arranged to seat the elastically deformable material.

14. A system according to claim 13 wherein the cap is arranged to be moveable relative to the shoulder thereby causing compression of the elastic material against the shoulder.

15. A system as hereinbefore described with reference to the accompanying drawings.
